(19) United States
(12) Patent Application Publication
(10) Pub. No.: US 2018/0220911 A1
(43) Pub. Date: Aug. 9, 2018

(54) BRAIN FUNCTION MEASURING APPARATUS

(71) Applicants: MIYUKI GIKEN Co., Ltd., Tokyo (JP); HIROSHIMA CITY UNIVERSITY, Hiroshima-shi (JP)

(72) Inventor: Osamu HIWAKI, Hiroshima-shi (JP)

(21) Appl. No.: 15/640,833

(22) Filed: Jul. 3, 2017

(30) Foreign Application Priority Data
Feb. 3, 2017 (JP) 2017-018219

(51) Int. Cl. A61B 5/04; A61B 5/00

(52) U.S. Cl. CPC A61B 5/04008; A61B 5/6819; A61B 2562/04; A61B 2562/0223; A61B 5/682

(57) ABSTRACT

The present invention provides a brain function measuring apparatus that includes a magnetic generator that is arranged at a deep portion of the brain of a subject and generates a magnetic field; and a magnetic sensor that is arranged at the scalp of the brain, and senses the magnetic field generated by the magnetic generator, wherein the magnetic sensor senses the magnetic field passed through the brain after being generated by the magnetic generator and relates to a brain activity of the subject.

### BACKGROUND OF THE INVENTION

### Field of the Invention

The present invention relates to a brain function measuring apparatus using means for generating magnetic field, wherein the means for generating magnetic field includes a magnet and the like.

### Description of the Prior Art

In recent years, a functional near-infrared spectroscopy (NIRS), for example, has been proposed to measure brain function. This method includes transforming visual and hearing information and the like which are inputted via sensory organ, e.g., eye, ear and the like to electrical signal, and measuring information transmission function of neuron as change of character of oxygenated hemoglobin (oxyHb) that flows in capillaries of brain during transmitting the electrical signal to the brain using near-infrared light.

Patent Document 1 (Japanese Patent Laid-Open S57-115232) and Patent Document 2 (Japanese Patent Laid-Open S62-275323) disclose an invention that relates to a brain activity measuring apparatus for measuring activity of the brain (brain activity) at the cerebral cortex of a subject using the above-mentioned method in which near-infrared light is utilized. The invention disclosed by Patent Document 1 and Patent Document 2 provides a measuring apparatus that irradiates near-infrared light from a near-infrared light source (or light source, for simplicity) that is arranged at the scalp of a subject to the brain of the subject, and receives reflection light and scattered light caused by the irradiation to the brain using a light receiver that is also arranged at the scalp of the subject for measuring change of blood flow in the brain.

Further, Patent Document 3 discloses an invention relating to a non-invasive brain activity measuring method that includes measuring near-infrared light that transmits (passes) through the brain of a subject. This invention includes arranging a near-infrared light source inside the oral cavity of the subject, irradiating near-infrared light in the scalp direction from the bottom portion of the brain of the subject, and receiving transmitted light by a light receiver arranged on the scalp for measuring change of blood flow in the brain. In this method, it is possible to measure brain function that is sensitive to the brain activity because the near-infrared light transmits through the deep portion of the brain.

However, in the measuring apparatus disclosed by the Patent Document 1 and 2 in which the near-infrared light source and the light receiver that receives reflection light and scattered light are arranged in the same scalp of the brain of the subject, a light path may be in the order of several centimeters, for example. Hence, the apparatus disclosed by the Patent Document 1 and 2 may have only low spatial resolution and may be capable to measure the brain activity only at the superficial portion of the brain, i.e., it is difficult to measure the brain activity at the deep portion of the brain with high resolution, for example. Further, the measuring apparatus disclosed by the Patent Document 1 and 2 may measure change in absorbance of oxygenated hemoglobin and deoxygenated hemoglobin (deoxyHb) in the order of at most a few seconds so that the measuring apparatus has low time resolution.

On the other hand, in the brain activity measuring apparatus according to the method disclosed by the Patent Document 3, because a near-infrared light source is arranged inside the oral cavity of a subject and electric power is supplied to the near-infrared light source for outputting near-infrared light via a lead, a signal line, and/or the like, it is necessary that the lead, the signal line, and/or the like is inserted into the oral cavity of the subject and a battery that supplies electric power to the near-infrared light is arranged in the oral cavity, so that the subject may have discomfort feeling and measurement work may become complicated. Further, a sensory organ that is located close to the bottom portion of the brain, for example, the cornea of the eye may have bad influence since near-infrared light may pass through the deep portion of the brain. Further, the light receiver may be expansive and the light receiver should be contacted to the scalp tightly. That is, when hair is inserted between the scalp and the light receiver, for example, the sensitivity of the brain activity measuring apparatus may be reduced and measurement work may become complicated.

### SUMMARY OF THE INVENTION

The present invention provides to solve the above-mentioned problem a brain function measuring apparatus that can not only measure brain function at the deep portion of the brain with high resolution, gives the minimum discomfort feeling to a subject and makes measurement work of the brain function easy, but also gives no bad influence on the cornea of the eye of the subject. Further, in the brain function measuring apparatus according to the present invention, there is no need to use any expensive light receiver and it is possible to use inexpensive and small magnetic sensor.

That is, the present invention provides a brain function measuring apparatus that includes a magnetic generator that is arranged at a deep portion of the brain of a subject and generates a magnetic field; and a magnetic sensor that is arranged at the scalp of the brain, and senses the magnetic field generated by the magnetic generator, wherein the magnetic sensor senses the magnetic field passed through the brain after being generated by the magnetic generator and relates to activity of the brain (brain activity) of the subject.

Further, the magnetic generator may be a magnet such as a neodymium magnet and the like, and the magnetic sensor may be composed of a plurality of one-axis magnetic sensors, two-axis magnetic sensors, three-dimensional magnetic sensors, or the like.

Further, the magnetic generator may be arranged inside the oral cavity or the nasal cavity of the subject, and the magnetic sensor may sense the magnetic field (magnetic flux) that is generated by the magnetic generator and relates to the brain activity.

### BRIEF DESCRIPTION OF THE DRAWINGS

FIG. 1 is an explanatory diagram of a brain function measuring apparatus according to the present invention;

FIG. 2 is a diagram that explains a structure of a personal computer (PC); and

FIG. 3 is a diagram that explains a sensed waveform sensed by a magnetic sensor and displayed on a display of the PC.

### DESCRIPTION OF THE PREFERRED EMBODIMENT

Hereinafter, embodiments of the present invention will be explained referring to the drawings.

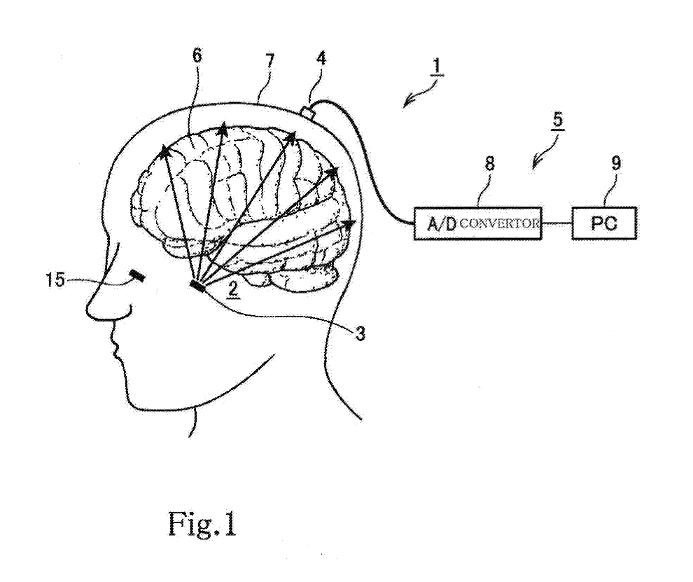

FIG. 1 is an explanatory diagram of a brain function measuring apparatus according to the present invention. In FIG. 1, the brain function measuring apparatus 1 includes a magnet 3 that is arranged at the bottom portion 2 of the brain of the subject, a magnetic sensor 4 that senses a magnetic field (or magnetism, a magnetic field signal) generated by the magnet 3, and a controller 5 that performs processing to estimate (measure) activity of the brain (hereinafter it will be referred to as brain activity) based on information about the magnetic field, for example, sensed by the magnetic sensor 4.

Here, the magnet 3 is a neodymium magnet, for example, and is arranged at the bottom portion 2 of the brain via a mouthpiece (not expressed with a figure), for example, so that the magnet 3 can be arranged at a predetermined position in the bottom portion 2 of the brain easily by the subject fixing the mouthpiece to the predetermined position in his/her mouth cavity when the measurement is carried out. Further, the magnet 3 is not limited to be the neodymium magnet, and any permanent magnet or electromagnet that has high magnetic flux density and has a potential to be downsized can be used as the magnet 3.

The magnetic sensor 4 is arranged on the scalp 7 of the brain of the subject. Although only a single magnetic sensor 4 is illustrated in FIG. 1, it is possible to use a plurality of magnetic sensors, for example, nineteen magnetic sensors by necessity for carrying out a standardized measurement, wherein each of the magnetic sensors should be arranged at a corresponding predetermined position. The installation of the magnetic sensor 4 may be done by using any useful method including adhering, coating, and the like. In the present example, linear-output magnetic field sensors can be used as the magnetic sensor 4 to sense magnetic field (magnetic field signal) at one of the points on the scalp 7 of the brain where the one of the linear-output magnetic field sensors is arranged, and the magnetic field sensed by the one of the linear-output magnetic field sensors is transformed to a voltage signal (value of voltage) for outputting information about the sensed magnetic field to a controller 5.

The controller 5 includes an A/D converter 8 and a personal computer (PC) 9. The A/D converter 8 transforms the voltage signal (value of voltage) to a corresponding digital signal and outputs it to the PC 9. The PC 9 performs processing to measure brain function of the subject based on information that relates to the digital signal that is generated by the A/D converter 8 and is sensed by the magnetic sensors 4, each of the magnetic sensors 4 being arranged at a predetermined position on the scalp of the brain, and performs analysis processing of the brain function.

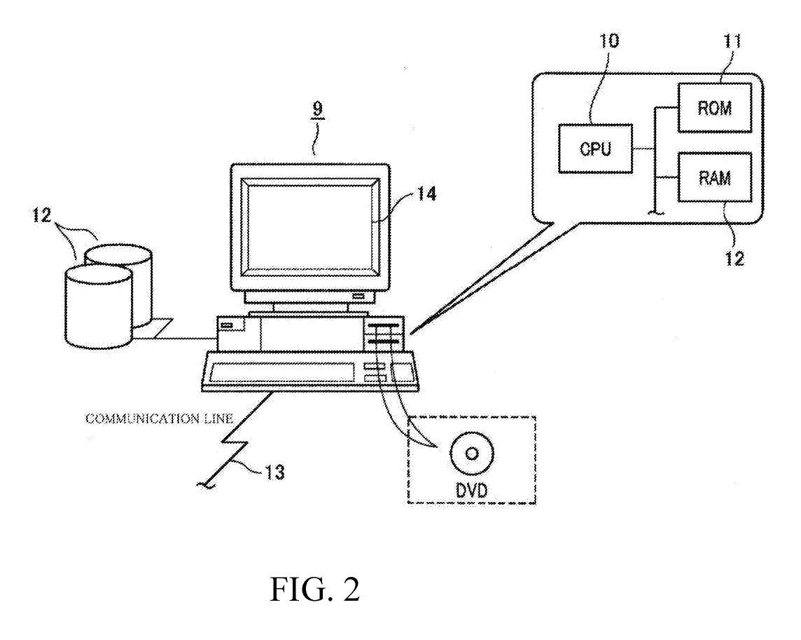

FIG. 2 is a diagram that explain a structure of a personal computer (PC) 9. The PC 9 includes a central processing unit 10, a read only memory (ROM) 11, random access memory (RAM) 12, and the like. The CPU 10 performs a processing referring to a system program stored in the ROM 11. For example, information about magnetic field sensed by the magnetic sensor 4 at a plurality of points on the scalp of the brain may be stored in a hard disk 12 that is connected to the PC 9, and the PC 9 performs analysis of the brain function of the subject, analysis of localization of the brain function and the like.

Further, a communication line 13 is connected to the PC 9 and the PC 9 performs processing to output a stimulus signal to the subject. On a display 14 of the PC 9, information about waveform of the magnetic field sensed by the magnetic sensor 4 and generated in respond to the stimulus signal is displayed.

Next, processing of measuring the brain function of the subject using the brain function measuring apparatus 1 that has the above-mentioned structure will be explained. At first, a mouthpiece in which the magnet 3 is fixed as discussed above, for example, is inserted to the mouth cavity of the subject such that the magnet 3 is set at a predetermined position on the bottom portion 2 of the brain as illustrated in FIG. 1. Under this situation, the magnet 3 generates magnetic flux of 0.1 Tesla, for example, to form a magnetic field in the brain of the subject spreading radically from the bottom portion 2 of the brain. It is widely known that magnetic field of order of 0.1 Tesla has no bad influence on the brain.

Next, the PC 9 performs processing to output a stimulus signal to the subject via the communication line 13 to give a predetermined stimulus to the subject, and the PC 9 performs processing to receive information about the magnetic field that the magnetic sensor 4 sensed. The magnetic flux generated by magnet 3 spreads radically from the bottom portion 2 of the brain where the magnet 3 is arranged as discussed above, penetrates all points in the brain of the subject, e.g., hippocampus, cerebral cortex, and the like, and then reach to the magnetic sensor 4 that is positioned beyond the hippocampus, the cerebral cortex, and the like of the brain. During the magnetic flux is penetrating through the brain, neuron and blood capillaries are influenced by the stimulus that may be generated by the magnetic flux, and the magnetic sensor 4 receives different level of the magnetic signal according to the position where the magnetic sensor 4 is arranged. Further, it is possible to measure steady-state brain activity without giving stimulus to the subject to measure and analyze the brain activity relating to mental state of the subject, feeling, effect of exercise, and the like.

Therefore, the magnetic sensor 4 that is arranged on the scalp of the brain receives magnetic field signal that has different character depending on the position where the magnetic sensor 4 that is arranged, and outputs information based on the magnetic field signal to the PC 9. The PC 9 performs processing to receive information from all of magnetic sensors 4 (for example, nineteen magnetic sensors 4 arranged at nineteen positions), and measures brain function based on the information.

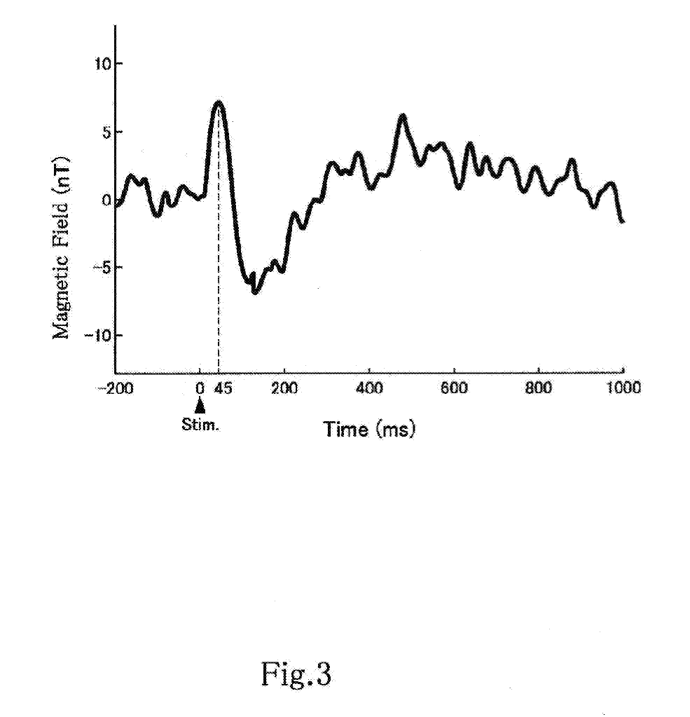

FIG. 3 is a diagram that explain a waveform of the magnetic field signal sensed by a magnetic sensor 4 and displayed on a display 14 of the PC 9. In FIG. 3, the horizontal axis is for elapsed time after the stimulus signal is outputted, and the vertical axis is for change of the level of the magnetic field that is received by the magnetic sensor 4. In the vertical axis, “Stim” indicates the timing when the stimulus signal is outputted.

As shown in FIG. 3, in the brain function measuring apparatus 1 according to the present example, because the magnetic field signal reaches a peak in a short time (for example, 45 milli-seconds) after the stimulus signal is outputted to the brain of the subject, it is possible to determine whether or not a portion of the brain of the subject where the magnetic sensor 4 is positioned is subject to the stimulus in real time. This waveform represents somatosensory evoked potential, and is based on the magnetic field signal of the magnetic sensor 4 located at a predetermined position. The similar measurement results can be obtained by all other magnetic sensors 4 arranged on the scalp of the brain of the subject. Therefore, the PC 9 can perform processing to carry out a brain function measurement on the subject in real time using the information that may be obtained by magnetic sensor 4, for example.

In FIG. 3, the waveform may be obtained by measurements in which the stimulus signals expose to a predetermined portion of living body of the subject several times with a specific interval between the neighboring stimulus signals and a specific time widths of each stimulus signal.

Therefore, the present example can provide the brain function measuring apparatus that can measure the brain function within a very short time and with high time resolution.

Further, in the brain function measuring apparatus according to the present example, because the magnet 3 is arranged at the bottom portion 2 of the brain, it is possible to measure the brain function in the wide range from the deep portion of the brain to the cerebral cortex, and to obtain an accurate result of the measurement with high precision.

Therefore, in the brain function measuring apparatus according to the present example, when a portion of the brain near the magnetic sensor where the level of the magnetic field signal may be large is specified to be a target portion of the measurement, it is possible to measure a localization of the brain activity with high precision. Hence, the brain function measuring apparatus according to the present example has high space resolution.

Further, the brain function measuring apparatus 1 according to the present example does not use near-infrared light and has no need to locate any lead and communication line for positioning a near-infrared light source in the mouth cavity of the subject, therefore the subject may have no bad feeling. The brain function measuring apparatus 1 according to the present example may be easy to use because the subject holds the mouthpiece in which the magnet 3 is fixed in his/her mouth cavity.

Further, because the brain function measuring apparatus 1 according to the present example does not use near-infrared light, it is possible to prevent the bad influence at the cornea of the subject from being occurred. That is, in the brain function measuring apparatus 1 according to the present example, the magnet 3 is arranged near the bottom portion 2 of the brain, wherein a sensory organ (eye) is near the bottom portion 2 of the brain, as shown in FIG. 1. If a near-infrared light source is arranged at the bottom portion 2 of the brain, it may be occurred that the cornea of the eye of the subject has bad influence by the near-infrared light. However, there is no need to worry about the bad influence being occurred at the cornea of the eye of the subject in the brain function measuring apparatus 1 according to the present example that does not use near-infrared light.

Further, in the brain function measuring apparatus 1 according to the present example, because the magnetic sensor 4 can be arranged on the scalp of the brain of the subject without any limitation of number, position and the like, it is possible that magnetic sensors 4 are arranged at high density so that the magnetic sensors 4 can measure the magnetic flux that has passed through the brain with minimum loss of information of the magnetic flux. Hence, it is possible to obtain the brain function measuring apparatus 1 that can sense the brain activity occurred at any position of the brain and can measure the brain function with high precision.

Further, the magnet 3 used in the present embodiment may have a lower price than any magnet used in any conventional example of the brain function measuring apparatus. In the brain function measuring apparatus according to the present example, there is not necessary that a light receiver is arranged to be contact with the scalp of the brain tightly. Further, for example, the brain function measuring apparatus can receive the magnetic field signal without any loss of information and can keep the high sensitivity even when a hair is inserted between the scalp of the brain and the magnetic sensor 4, so that the brain function measuring apparatus 1 according to the present example may become easy to use for measurement work.

Further, the magnet 3 is arranged in the mouth cavity of the subject in the explanation of the present embodiment. However. Is is possible that the magnet (for example, a magnet 15) is arranged in the nasal cavity of the subject. In such structure of brain function measuring apparatus, it is possible to measure the brain function in a wide range from the deep portion of the brain to cerebral cortex, and to obtain an accurate result of the measurement with high precision.

Further, in the explanation of the present embodiment, the magnet has the magnetic flux of order of 0.1 Tesla. However, it is possible to use any magnet being able to generate a different level of the magnetic flux other than 0.1 Tesla. Further, it is possible to use a magnet that has a smaller size and smaller magnetic force and is made of a material having high permeability, such as silicon system to increase the magnetic flux in the direction to the brain.

Further, it is possible to use a three-dimensional magnetic sensor as the magnetic sensor 4 to estimate a portion of the brain where the brain activity occurs with high precision.

### EXPLANATION OF THE SYMBOLS

1 brain function measuring apparatus
2 bottom portion of the brain of a subject
3 Magnet
4 magnetic sensor
5 controller
6 brain of the subject
7 scalp of the brain
8 A/D convertor
9 personal computer (PC)
10 central processing unit (CPU)
11 read only memory (ROM)
12 random access memory (RAM)
13 communication circuit
14 display
15 magnet

1. A brain function measuring apparatus, comprising:
a magnetic generator that is arranged at a deep portion of the brain of a subject and generates a magnetic field; and
a magnetic sensor that is arranged at the scalp of the brain, and senses the magnetic field generated by the magnetic generator,
wherein the magnetic sensor senses the magnetic field passed through the brain after being generated by the magnetic generator and relates to a brain activity of the subject.

2. The brain function measuring apparatus according to claim 1, wherein
the magnetic generator is a neodymium magnet.

3. The brain function measuring apparatus according to claim 2, wherein
the magnetic sensor comprises a plurality of magnetic sensors arranged on the scalp of the brain.

4. The brain function measuring apparatus according to claim 2, wherein
the magnetic sensor comprises a plurality of three-dimensional magnetic sensors arranged on the scalp of the brain.

5. The brain function measuring apparatus according to claim 2, wherein
the magnetic generator is arranged inside the oral cavity of the subject.

6. The brain function measuring apparatus according to claim 2, wherein
the magnetic generator is arranged inside the nasal cavity of the subject.

7. The brain function measuring apparatus according to claim 1, wherein
the magnetic sensor comprises a plurality of magnetic sensors arranged on the scalp of the brain.

8. The brain function measuring apparatus according to claim 1, wherein
the magnetic sensor comprises a plurality of three-dimensional magnetic sensors arranged on the scalp of the brain.

9. The brain function measuring apparatus according to claim 1, wherein
the magnetic generator is arranged inside the oral cavity of the subject.

10. The brain function measuring apparatus according to claim 1, wherein
the magnetic generator is arranged inside the nasal cavity of the subject.
